(19) United States
(12) Patent Application Publication
(10) Pub. No.: US 2004/0193448 A1
(43) Pub. Date: Sep. 30, 2004

(54) Touch-screen applications for outpatient process automation

(76) Inventors: Peter Woodbridge, Indianapolis, IN (US); Michael Lay, Saline, MI (US); Gary Moore, Indianapolis, IN (US)

Correspondence Address: DYKEMA GOSSETT PLLC, Suite 300 West, 1300 I Street, N.W., Washington, DC, 20005, US

(21) Appl. No.: 10/636,772

(22) Filed: Aug. 8, 2003

Related U.S. Application Data
(60) Provisional application No. 60/402,734, filed on Aug. 13, 2002.

(51) Int. Cl. G06F 17/60; G09B 19/00; A61B 5/00

(52) U.S. Cl. 705/2; 434/118; 600/300

(57) ABSTRACT

A method and apparatus for HIPAA compliant outpatient process automation is provided. The method includes prompting a user to select at least one anonymous or patient specific application. If the user selects an anonymous application, the method includes receiving from the user anonymous query information and matching the anonymous query information to anonymous information stored in a database, and thereafter displaying guidance based upon the matched anonymous information. If the user selects a patient specific application, the method includes receiving from the user patient specific query information and matching the patient specific query information to patient specific information stored in a database, and thereafter displaying guidance based upon the matched patient specific information. The patient specific information may include a medical record information application, an appointment information application, a questionnaire and survey information application, an education information application, a symptom information application, and a risk assessment information application.

### RELATED APPLICATIONS

This application claims benefit of priority of Provisional Application Serial No. 60/402,734, filed Aug. 13, 2002.

### BACKGROUND OF INVENTION

a. Field of Invention

The invention relates generally to outpatient healthcare and systems for automation thereof, and, more particularly to a computerized apparatus and method for automatically tracking, updating and managing outpatient care.

b. Description of Related Art

Veterans earn the right to healthcare through their service in the military. This entitlement has intended and unintended consequences. While the intended consequences are apparent, the unintended consequences mean that the Veterans Health Administration (VHA) can not deny care to eligible veterans, VHA must take care of all patients regardless of cost of care or behavioral problems, medical centers can not avoid non-profitable patients by providing only certain types of care, nor can the VHA avoid caring for veterans that other health systems do not want—e.g. the homeless, patients with severe mental illness, and patients with substance abuse problems. Unintended consequences of healthcare entitlement are more insidious, in that a patient's expectations are frequently inappropriate. Patients often demand Veteran's Affairs (VA) healthcare providers to prescribe non-formulary drugs when prescribed by an outside (non-VA) provider. Additionally, when healthcare is viewed as an entitlement, patients have little incentive to participate actively in their own care and comply with treatment regimens and make necessary lifestyle changes. This adds to healthcare costs and abrogates much of the benefit of taking care of patients “for-life.”

These exemplary demands for care of veterans are consistent with the problems faced by providers who service non-military patients, and generally result in an healthcare provider exceeding appropriated funds or yearly healthcare budgets. Since healthcare providers often have limited authority in collecting non-appropriated funds or exceeding yearly budgets, providers often resort to other means of investing funds in activities that generate maximum value for their customers. One strategy for reducing inappropriate demands on the provider is to educate patients to become “informed consumers.” Research performed herein has shown that patients are in fact interested in becoming better-informed about their healthcare, and that they do not receive enough information about their care.

For example, the Dallas VAMC employed the “Howdy” automated check-in program to speed up check-in in phlebotomy by enabling patients with lab orders to bypass the check-in window. Shortly after the Dallas VAMC implemented the “Howdy” program, they noted a significant increase in “missed orders”—patients checking into phlebotomy with no orders. Initially, the thought was that something had gone amiss in the clinic order entry process. On further investigation, it became apparent that patients were simply swiping in to see if there were any pending orders. In other words, patients simply wanted more information about their healthcare.

In the past, healthcare providers have realized that as informed consumers, patients develop the knowledge and skills to take a more active role in their own care. This strategy, which has been employed in the past, is known as shared-care, and generally involves patients participating and taking responsibility for the portion of their care that they control. Not only did shared-care improve customer satisfaction over the years, it also improved compliance with therapeutic regimen and thereby reduced costs. For example, compliance to therapeutic regimens also improved when patients understood the nature of their illness, why they were being treated, how long the treatment would result, and the anticipated results of the treatment. In other words, shared-care provided patients with enough information to be active participants in their own care.

Since most shared-care strategies involve patient education, outpatient providers and nurses are often required to educate patients. For providers employing a shared-care strategy, additional staff is required for patient education, since providers and nurses generally do not have the time for patient education. The additional staff directly results in an increase in a providers operating costs, and therefore exemplifies the need for another automation strategy that facilitates patient education for further enabling shared care.

One such automation strategy involves the use of touch-screen technology, which has been used over the years to facilitate data management without the need for a separate keyboard or other such devices. While touch-screen technology has found applications in grocery stores and fast food stores, for example, for facilitating the checkout process, in the healthcare industry, touch-screen technology has been limited largely to data entry by professional staff on dedicated devices or into anonymous patient information “kiosks.”

With regard to anonymous patient information kiosks, since access is anonymous, healthcare providers are unable to determine which patient is receiving education, and whether or not the education is relevant to the patient's specific medical condition.

With regard to touch-screen technology available to healthcare professionals, such technology is often limited in purpose to the management of patient healthcare data, such as an individual's identifying information, allergy or prescription information, as well as past or future appointment records. Thus the focus of technology available to healthcare professionals is directed primarily toward legal, monetary and scheduling concerns, and not toward the actual management of an individuals healthcare needs.

Due to the aforementioned limitations of technology available to patients, patients often play a limited role in their own healthcare management, largely in the form of completing individual questionnaires to allow a healthcare provider to thereafter analyze and manage the individual's healthcare needs. Such management techniques often lead to a disconnect between a patient's ability to manage their individual healthcare, and the healthcare provider's ability to efficiently diagnose the patient, since a significant portion of the provider's time and resources that can be devoted to a single patient are in fact spent performing data entry or other such rudimentary tasks. For large healthcare providers, usage of resources in performing data entry and other such tasks can lead to a loss of millions of dollars in valuable revenue each year, which resources could in turn have been applied to systems for improving patient healthcare or for reducing healthcare fees.

In today's economy, healthcare enterprises are struggling with burgeoning expenses and a relatively flat income. Consequently there is developing interest among healthcare providers and intermediaries in demand management. Shared-care demand strategies, although proven effective, in numerous studies have failed to achieve wide acceptance due to the high labor cost of implementation.

Accordingly, there remains a need for a healthcare management system, which enables patients to “self-serve” their individual healthcare needs, thereby improving customer service and also reducing cost of healthcare delivery. There also remains a need for a healthcare management system which provides a cost-effective method for not only automating required data gathering activities, but also mass-customizing shared-care demand management.

### SUMMARY OF INVENTION

The invention solves the problems and overcomes the drawbacks and deficiencies of the prior art healthcare management systems by providing a touch-screen application for outpatient automation which provides a cost-effective method for not only automating required data gathering activities, but also provides mass-customizing shared-care demand management.

Another aspect of the present invention is to provide a touch-screen application system, which meets the privacy and security requirements for compliance with the Health Insurance Portability and Accountability Act of 1996 (HIPAA).

Yet another aspect of the present invention is to provide a touch-screen application for providing and documenting patient education for Joint Commission on Accreditation of Healthcare Organizations (JCAHO) and other regulatory body purposes.

Another aspect of the present invention is to provide a touch-screen application for automating patient access to information about their clinical condition so as to improve patient satisfaction, and reduce overall healthcare provider costs by reducing demand for ineffective healthcare and by improving disease management.

The present invention achieves the aforementioned exemplary aspects by providing a method for outpatient process automation. The method includes the step of prompting a user to select at least one anonymous or patient specific application. If the user selects the anonymous application, the method includes receiving from the user anonymous query information, matching the anonymous query information to anonymous information stored in a database, and displaying guidance based upon the matched anonymous information. If the user selects the patient specific application, the method includes receiving from the user patient specific query information, matching the patient specific query information to patient specific information stored in a database, and displaying guidance based upon the matched patient specific information. User access to the patient specific application may be controlled by a secure log-in process.

For the method described above, the anonymous application may include at least one of way-finding information, general healthcare provider information, eligibility and benefits information, and staff directory information for healthcare providers. The secure log-in process for access to the patient specific application may include prompting the user to log-on by entering a social security number by either touching a displayed number pad or sliding an identification card through an attached card reader, and prompting the user to enter a password. If the log-in is successful, the method includes providing the user access to the patient specific application, and if the log-in is unsuccessful, the method includes assisting the user to obtain access to the patient specific application. The assisting aspect of the method described above may include verifying that a system for implementing the method is operational. If the system is operational and the user is a registered user, the method includes determining whether the user password and identification card are operational, if not, then the method includes assisting the user to obtain a new password or identification card, if so, then the method includes releasing requested information to the user. If the system is operational and the user is a non-registered user, the method includes assisting the user to log-in and register into the system by means of the social security number. If the system is non-operational, the method includes moving the user to another system for implementing the method. The providing aspect of the method described above may include controlling release of patient information in accordance with HIPAA regulations by requiring the user to authorize display of patient specific application data through a signed release of information (ROI). The providing aspect may further include, prompting the user to select at least one of a medical record information application, an appointment information application, a questionnaire and survey information application, an education information application, a symptom information application, and a risk assessment information application. The medical record information application may include at least one of a problem list with links to education material, medication list with links to education material, recent laboratory results with links to education material, pending orders with links to preparation material, and means for enabling a user to order medication refills. The appointment information application may enable the user to at least one of look up and change future appointments for a healthcare provider. The questionnaire and survey information application may enable the user to at least one of answer and enter data directly into the system, enabling means testing and enabling measurement of healthcare effectiveness. The education information application may enable the user to obtain information on at least one of medical conditions and general medical information. The symptom information application may enable the user access to software that provides symptom triaging. The risk assessment information application may enable the user access to health-screening tools.

The invention also provides a system for outpatient process automation. The system includes means for prompting a user to select at least one anonymous or patient specific application, means for selecting the anonymous application, means for selecting the patient specific application, and means for controlling access to the patient specific application by a secure log-in process. For the anonymous application, the system may receive from the user anonymous query information, match the anonymous query information to anonymous information stored in a database, and display guidance based upon the matched anonymous information, and for the patient specific application, the system may receive from the user patient specific query information, match the patient specific query information to patient specific information stored in a database, and display guidance based upon the matched patient specific information.

For the system described above, the anonymous application may include at least one of way-finding information, general healthcare provider information, eligibility and benefits information, and staff directory information for healthcare providers. The secure log-in process for access to the patient specific application may include means for prompting the user to log-on by entering a social security number by either touching a displayed number pad or sliding an identification card through an attached card reader, and means for prompting the user to enter a password. If the log-in is successful, the system may include means for providing the user access to the patient specific application, if the log-in is unsuccessful, the system may include means for assisting the user to obtain access to the patient specific application. The assisting aspect of the system described above may include means for verifying that the system is operational, if the system is operational and the user is a registered user, the system may include means for determining whether the user password and identification card are operational, if not, then the system may include means for assisting the user to obtain a new password or identification card, if so, then the system may include means for releasing requested information to the user. If the system is operational and the user is a non-registered user, the system may include means for assisting the user to log-in and register into the system by means of the social security number. If the system is non-operational, the system may include means for moving the user to another system for implementing the method. The providing aspect of the system described above may include means for controlling release of patient information in accordance with HIPAA regulations by requiring the user to authorize display of patient specific application data through a signed release of information (ROI). The providing aspect of the system described above may further include means for prompting the user to select at least one of a medical record information application, an appointment information application, a questionnaire and survey information application, an education information application, a symptom information application, and a risk assessment information application.

Additional features, advantages, and embodiments of the invention may be set forth or apparent from consideration of the following detailed description, drawings, and claims. Moreover, it is to be understood that both the foregoing summary of the invention and the following detailed description are exemplary and intended to provide further explanation without limiting the scope of the invention as claimed.

### BRIEF DESCRIPTION OF THE DRAWINGS

The accompanying drawings, which are included to provide a further understanding of the invention and are incorporated in and constitute a part of this specification, illustrate preferred embodiments of the invention and together with the detail description serve to explain the principles of the invention. In the drawings:

FIG. 1 is a flow chart of a process, consistent with this invention, for accessing anonymous or patient specific touch-screen applications;

FIG. 2 is a flow chart of a process, consistent with this invention, for log-in of a patient for access to patient specific tough-screen applications;

FIG. 3 is a flow chart of a process, consistent with this invention, for failed log-in of a patient;

FIG. 4 is a flow chart of a process, consistent with this invention, for patient registration;

FIG. 5 is a flow chart of a process, consistent with this invention, for providing a patient access to patient specific touch-screen applications;

FIG. 6 is a flow chart of a process, consistent with this invention, for assessing a patient's pain status; and

FIG. 7 is an exemplary diagram of a network employing the touch-screen application system for outpatient process automation according to the present invention.

### DETAILED DESCRIPTION OF THE PREFERRED EMBODIMENTS

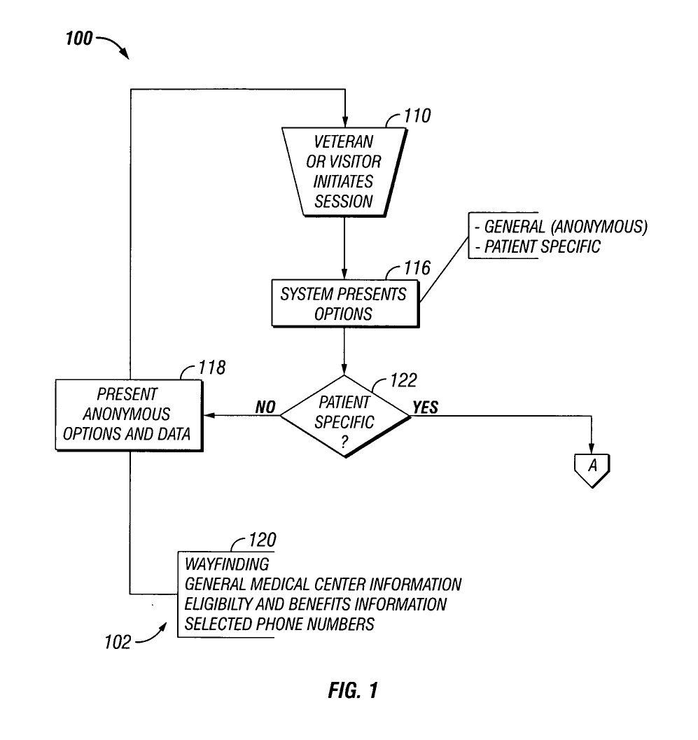

Referring now to the drawings wherein like reference numerals designate corresponding parts throughout the several views, FIGS. 1-7 illustrate components and processes of a touch-screen application system for outpatient process automation, hereinafter designated “touch-screen application system 100”, according to the present invention.

Before proceeding further with the detailed description of touch-screen application system 100, a brief history of the privacy and security requirements for compliance with the Health Insurance Portability and Accountability Act of 1996 (HIPAA) will be discussed for setting forth the necessary parameters for touch-screen application system 100 according to the present invention.

HIPAA, Public Law 104-191, was enacted on Aug. 21, 1996. Sections 261 through 264 of HIPAA required the Secretary of the U.S. Department of Health and Human Services (HHS) to publicize standards for the electronic exchange, privacy and security of health information. HIPAA required the Secretary to issue privacy regulations governing individually identifiable health information, if Congress did not enact privacy legislation within three years of the passage of HIPAA. Since Congress did not enact such privacy legislation, HHS developed a proposed rule and released it for public comment on Nov. 3, 1999. After receiving numerous public comments, HHS modified and re-published the Privacy Rule in Dec. 28, 2000. In March 2002, HHS proposed and released for public comment modifications to the Privacy Rule, and after receiving public comments, published final modifications to the Privacy Rule in Aug. 14, 2002.

The Privacy Rule standards, which are now in effect, address the use and disclosure of an individual's health information, denoted “protected health information” by organizations subject to the Privacy Rule, denoted “covered entities,” as well as standards for an individual's privacy rights to understand and control how their health information is used. Such “protected health information” includes all “individually identifiable health information” held or transmitted by a covered entity or its business associate, in any form or media, whether electronic, paper, or oral. Examples of “individually identifiable health information” include demographic data related to an individual's past, present or future physical or mental health or condition, the provision of healthcare to the individual, or the past, present, or future payment for the provision of healthcare to the individual, and that identifies the individual or for which there is a reasonable basis to believe can be used to identify the individual. Individually identifiable health information includes many common identifiers (i.e. name, address, birth date, Social Security Number). Thus, a major goal of the Privacy Rule is to assure that an individual's health information is properly protected while allowing the flow of health information needed to provide and promote high quality healthcare and to protect the public's health and well being.

The Privacy Rule covers health plans, healthcare providers and healthcare clearinghouses. Health plans include, for example, health, dental, vision, and prescription drug insurers, health maintenance organizations (“HMOs”), Medicare, Medicaid, Medicare+Choice and Medicare supplement insurers, and long-term care insurers (excluding nursing home fixed-indemnity policies). With regard to healthcare providers, the Privacy Rule covers every healthcare provider, regardless of size, who electronically transmits health information in connection with certain transactions. Lastly, healthcare clearinghouses include entities that process nonstandard information they receive from another entity into a standard (i.e. standard format or data content), or vice versa.

A major purpose of the Privacy Rule is to define and limit the circumstances in which an individual's protected heath information may be used or disclosed by covered entities. Thus, a covered entity may not use or disclose protected health information, except either as the Privacy Rule permits or requires, or as the individual who is the subject of the information (or the individual's personal representative) authorizes in writing. Specifically, a covered entity is permitted, but not required, to use and disclose protected health information, without an individual's authorization, to the individual (unless required for access or accounting of disclosures), for treatment, payment, and healthcare operations, after the individual has been given an opportunity to agree or object, incident to an otherwise permitted use and disclosure, for public interest and benefit activities, and for the purposes of research, public health or healthcare operations.

Based upon the aforementioned HIPAA privacy requirements, in general, the present invention comprises touch-screen application system 100 which enables a user secure HIPAA compliant, direct patient medical record access and automates certain patient information data entry functions. This “self-serve” capability is achieved through computerized touch-screen applications that require no user computer knowledge or expertise. Card readers and biometrics, specifically fingerprint identification, may be used for log-in and security, and touch-screen computer monitors may be used for navigation and data entry. Server based applications using Internet protocols enable consistency, scalability, and ease of maintenance, and multiple levels of security and release of information applications ensure HIPAA compliance.

Due to the patient's ability to “self-serve” certain aspects of healthcare delivery, touch-screen application system 100 encourages patients to more actively participate in their own care. Through touch-screen self-serve applications provided in system 100, patients are empowered to contribute to their own care, thereby improving customer satisfaction and reducing overall healthcare delivery costs. Greater patient satisfaction and cost reduction are achieved by, for example, allowing patients to securely and confidentially view limited portions of their medical records, thereby addressing patient needs to know more about their care, facilitating and automatically documenting patient education activities, thereby enabling shared-care and addressing patient needs to receive more information regarding their illnesses, medications and tests, and having patients enter certain information directly into their electronic medical records, thereby eliminating the cost of staff data entry.

Touch-screen application system 100, which meets the aforementioned HIPAA Privacy Rule guidelines, will now be described in detail.

Specifically, the present invention provides touch-screen application system 100, which enables a user secure HIPAA compliant, direct patient medical record access and automates certain patient information data entry functions. In the exemplary embodiment of FIGS. 1-7, touch-screen application system 100 may include four types of general application categories, including anonymous applications 102, self-administration questionnaire applications 104, patient specific information applications 106 and patient specific education applications 108. It should be noted that the designations visitor, veteran, patient or user, as used throughout this disclosure and in FIGS. 1-7, refer to a person using touch-screen application system 100.

Anonymous applications 102 may generally include way-finding and general patient assistance information. Self-administration questionnaire applications 104 may generally automate patient data capture, and include applications including regulatory required assessments (i.e. pain questionnaire), inventories (i.e. patient history, annual patient questionnaires), outcomes measures, and financial information (i.e. third-party insurance, Veterans Affairs means test). Patient specific information applications 106 may generally permit patients to display future appointments and enable patients access to limited extracts from their medical records. Accessible elements in a patient's records may include an appropriately redacted problem list, current medications, recent laboratory tests, and pending orders. Patient specific education applications 108 may not only link elements in the patient record extract to context specific educational materials, but also provide patients access to health screening and automated triage tools. Access to education applications 108 may be automatically documented in the patient's record to satisfy patient care and regulatory requirements. The responses in each of the aforementioned applications may be automatically incorporated into the individual patient's medical records.

Referring to FIG. 1, while most of the self-serve functions of the touch-screen application system 100 may require secure patient identification, anonymous applications 102 may be configured for access without secure patient identification. Anonymous applications 102 may provide for way-finding operations, access to general healthcare provider (i.e. healthcare provider) information and limited staff directory, and for the ability to complete application forms electronically. Specifically, for way-finding operations, anonymous applications 102 may provide a graphical tool for illustrating to a user a route to a specific destination in a healthcare provider. Through touch-screen pop-ups, a user may be queried as to their desired destination. Anonymous application 102 may then graphically illustrate the best route(s) by providing a map and text instructions, which could then be printed by the user to find his or her way around a healthcare provider.

With regard to access to general healthcare provider information, anonymous application 102 may also provide Web based brochures and informational pamphlets for patients and visitors for describing features and functions of the healthcare provider. In addition anonymous application 102 may provide information to patients and visitors informing them of their rights and entitlements. A directory with phone numbers and way-finding links may be provided to assist patients and visitors in contacting selected individuals and departments (i.e. customer service, health benefits unit). With regard to application completion, anonymous application 102 may provide new or pre-existing patients and visitors with the ability to complete forms electronically. An attached signature pad may be used to capture an electronic image of the patient's signature, and the completed form may then be securely transmitted to the healthcare provider.

As illustrated in FIGS. 1 and 7, in order to initiate the session for anonymous application 102, at block 110 a patient or visitor may simply touch screen 112 of workstation 114 to begin the session. For ease of use and navigation, all touch-screen applications may contain certain common elements, and include buttons that permit users to escape to the start screen and to back up one screen. For consistency, buttons that execute similar functions may be designated to have similar colors, shapes, and screen locations.

Upon touching screen 112, at block 116, touch-screen application system 100 may display a welcoming message (not shown) along with buttons representing anonymous application 102 as well as a single button that serves as a portal to the patient specific applications 104, 106 and 108.

Assuming that the user selects anonymous application 102, touch-screen application system 100 may access way-finding application 120 via path 118. As discussed above, in way-finding application 120, a patient or visitor may be able to find their way about a healthcare provider. By picking a specific destination, for example, a cardiology provider, the user may be prompted for further specificity, such as, heart, cardiology, or congestive heart failure, and then prompted to select his or her destination. Touch-screen application system 100 may then display a map with a best route overlay and instructions. Other anonymous applications such as general healthcare provider information, benefits and eligibility information, and a limited directory may also be accessed in anonymous application 102 and function in a similar manner as discussed above for way-finding application 120.

If instead of selecting anonymous application 102, the user selects patient specific application button 122, touch-screen application system 100 may access applications 104, 106 and 108 via Loop-A.

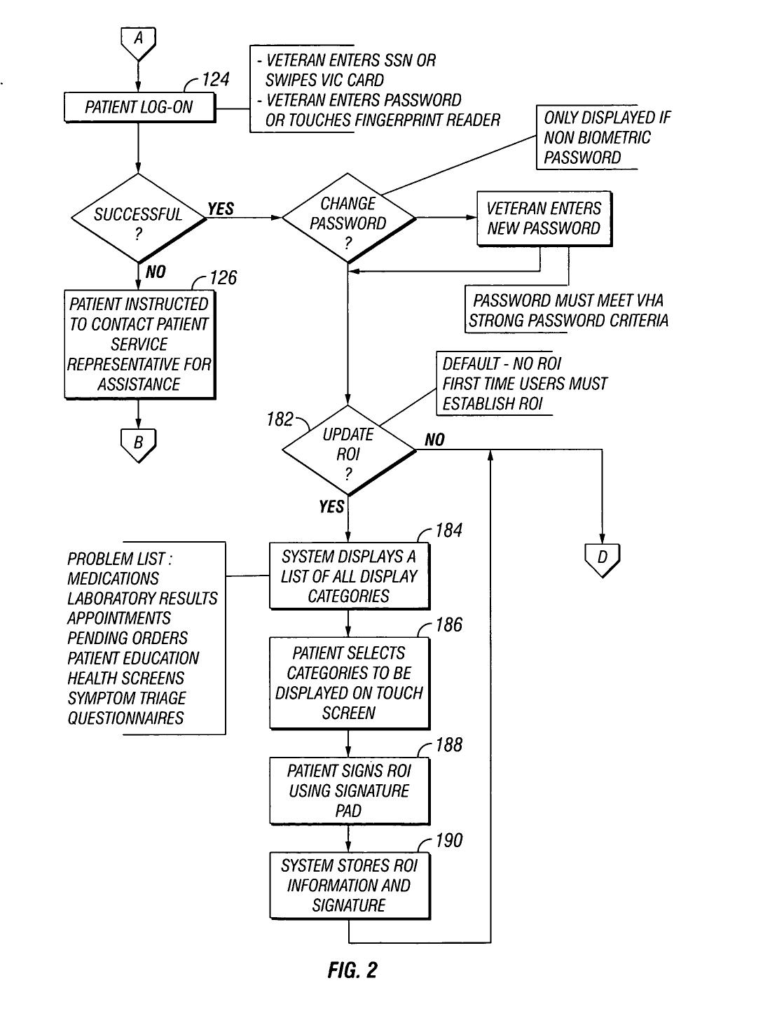

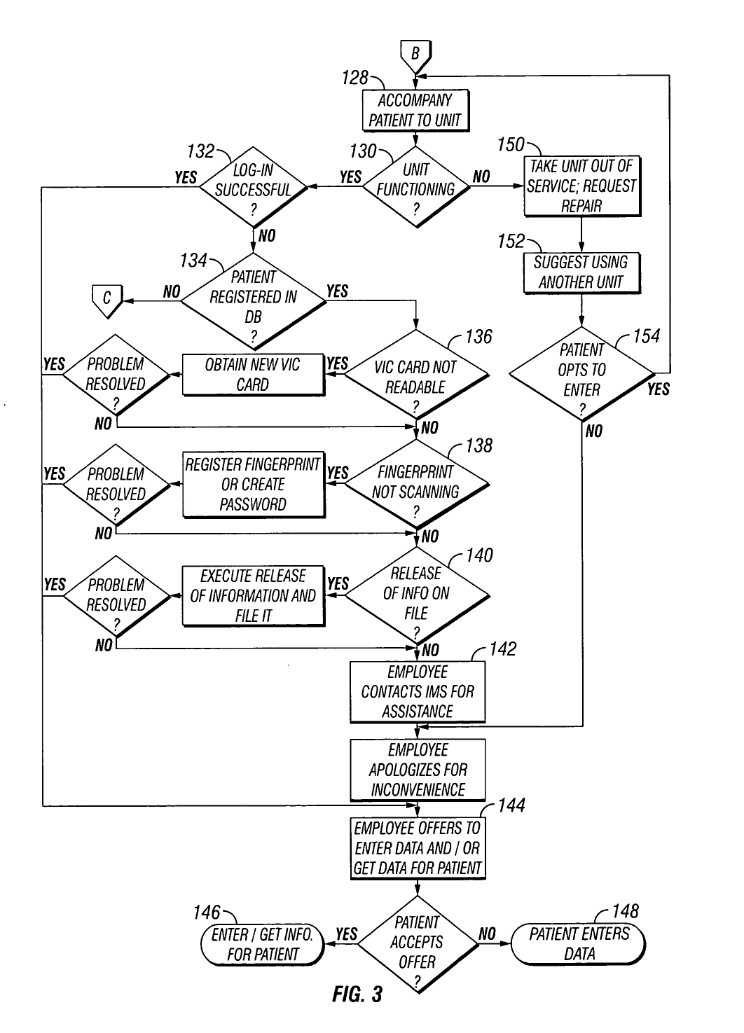

Referring to FIG. 2, for Loop-A, before entering applications 104, 106 and 108, a patient may be required to log-on by entering his or her social security number at block 124 by either touching a number pad displayed on the screen or sliding his or her identification card through an attached card reader. After entering the social security number, the patient may be prompted of his or her password, also at block 124. The patient can enter his or her password by either touching the appropriate keys on a displayed keyboard or by placing his or her finger on a fingerprint biometrics device (not shown). Patients who attempt to log-in and who are denied access for any other reason may be instructed to see provider support staff (i.e. an ambassador) at block 126. The provider ambassador may assist the patient with resolving the problem in accordance with the algorithms summarized in FIG. 3 for Loop-B.

Specifically, referring to FIG. 3, ambassador trouble shooting may include accompanying the patient to a workstation 114 at block 128, verifying that workstation 114 is functioning at block 130, and if so, that the log-in was successful at block 132, that the patient is registered in system 100 at block 134, that the patient's identification card and fingerprint are scannable at blocks 136 and 138, respectively, and thereafter releasing the information on file at block 140. In the event that the ambassador is unable to resolve the problem, the ambassador may assist the patient in obtaining relevant information. If none of the aforementioned trouble shooting steps are successful, at block 142, the ambassador may contact a service specialist for assistance. The ambassador may thereafter offer to enter data for the patient at block 144, and either enter patient data at block 146 or allow the patient to enter data at block 148 as needed. Alternatively, if the ambassador verifies that workstation 114 is not functioning at block 130, workstation 114 may be repaired at block 150 or the patient may be directed to another workstation at block 152 to thereafter enter data at block 154.

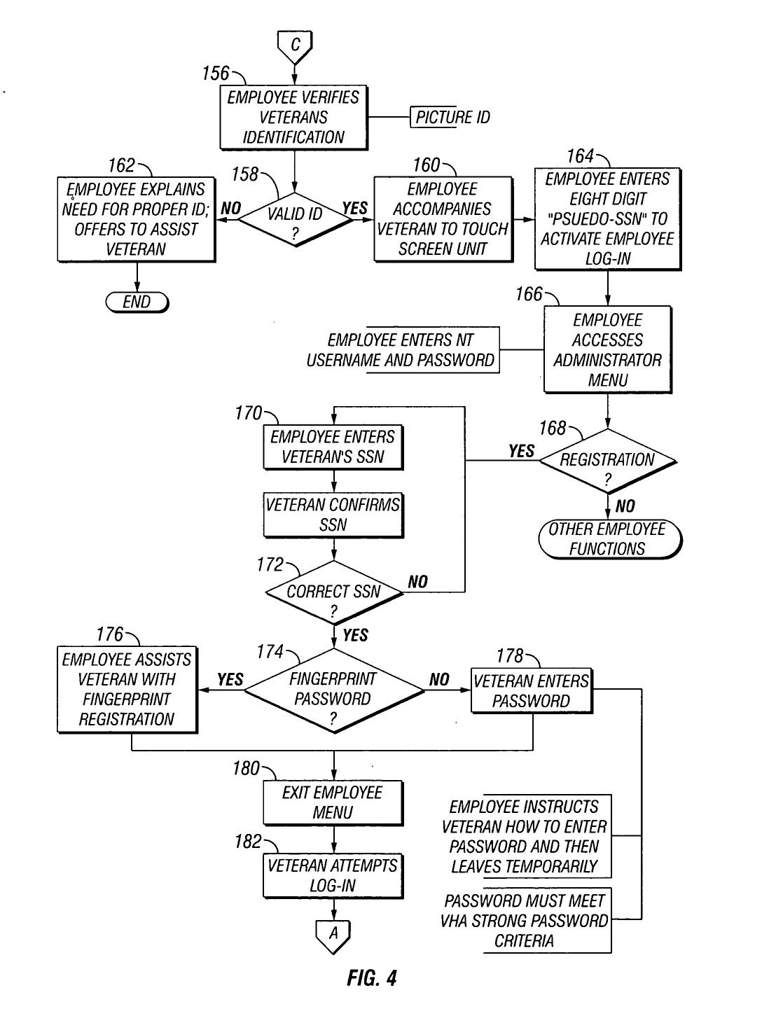

Referring to FIGS. 3 and 4, in the event the user is a first time user (i.e. not registered in system 100 at block 134), at Loop-C, a provider staff member may register the user at any available touch-screen workstation 114. As a first step, at block 156, the staff member may verify the patient's identification by examining a picture ID (i.e. driver's license, or a special provider identification card). After verifying the patient's identification at block 158, the employee (i.e. staff member) may log into the system at block 160. If the employee is unable to verify the patient's identification, the employee may explain the need for a proper identification and assist the patient accordingly at block 162. At block 164, the employee may access the employee login screen by entering a common eight-digit access code in lieu of a nine-digit social security number. Touch-screen application system 100 may branch to an employee login screen upon entry of the eight-digit employee code number. At the employee login screen at block 166, system 100 may prompt for the employee's NT username and password. The employee may enter his or her username and password on a displayed touch keyboard. At block 168, system 100 may then authenticate the user and display the employee menu. All employee access and actions may be tracked and recorded.

To register the patient, the employee may select a “register patient” button. The same button may be used to re-register a patient in the event that the patient forgets his or her password. The employee may enter the patient's social security number at block 170. After verification of the patient's social security number at block 172, the employee may then ask the patient if he or she wants to use his or her fingerprint as a password at block 174. If the patient agrees, the employee may assist the patient in registering his or her fingerprint at block 176. If the patient declines, he or she may be asked to create a strong password at block 178. After the patient is instructed on how to enter a password, the employee may step out of line of site of the touch-screen, and the patient may be asked to enter his or her password in system 100. Once the patient has established a strong password, the system may automatically exit the registration menu at block 180 and the patient may be invited to log-in at block 182 to gain access to applications 104, 106 and 108 at Loop-A.

Referring to FIG. 2, at Loop-A, assuming that the patient successfully logs-in and is given access to applications 104, 106 and 108, for each of the aforementioned applications of touch-screen application system 100, since release of patient information is in accordance with HIPAA regulations, patients using patient specific information applications are granted the right to determine what information is displayed on touch-screens 112. Since a risk exists that someone other than the patient may see data displayed on screens 112, patients must authorize the system to display data through a signed release of information (ROI), as illustrated in block 182 of FIG. 2. Specifically, at block 184, system 100 may ensure compliance by listing the categories of information that the patient has authorized the system to display and then ask the patient if he or she would like to change the options. First time users may not have any category marked as active. At block 186, first time users and users that want to change their options may be presented with a list of all categories of information that can be displayed. The patient may be asked to touch a “yes” button by all categories that he or she wants to authorize for display. Display categories may include, for example, problem list medications, laboratory results, pending orders, future appointments, health screens, questionnaires, and education. Once the patient has selected display categories, a release of information statement may be displayed. The patient may have the option of printing the release of information statement. At block 188, he or she may be asked to confirm the display options and indicate his or her agreement with the release of information statement by signing an attached signature capture device (not shown). Once the patient has signed the release of information, system 100 may store the release of information at block 190 and present applications that display authorized categories of information for applications 104, 106 and 108 at Loop-D.

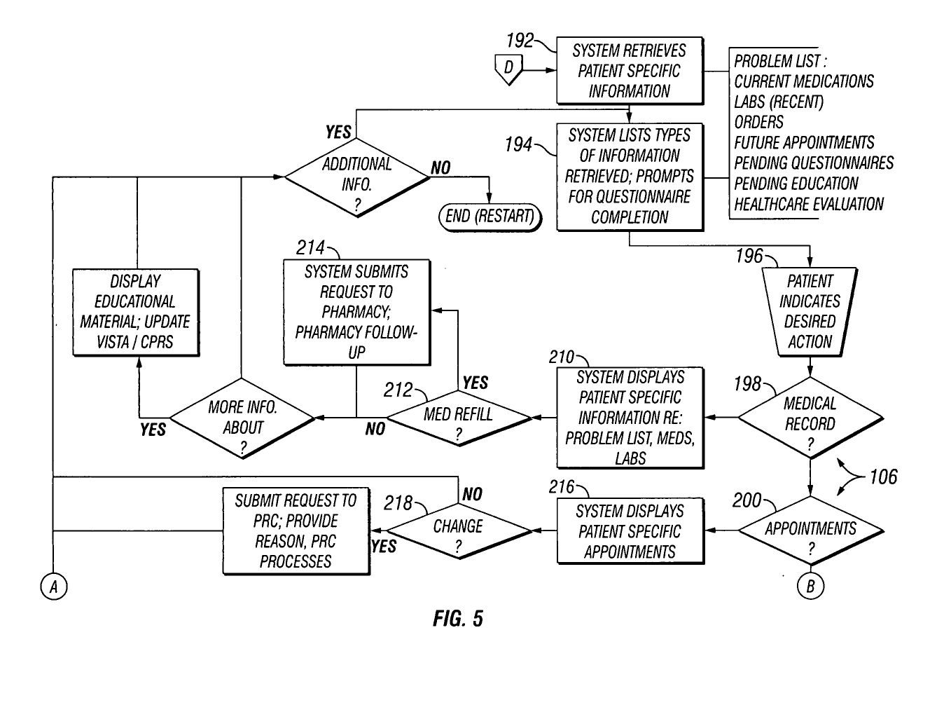

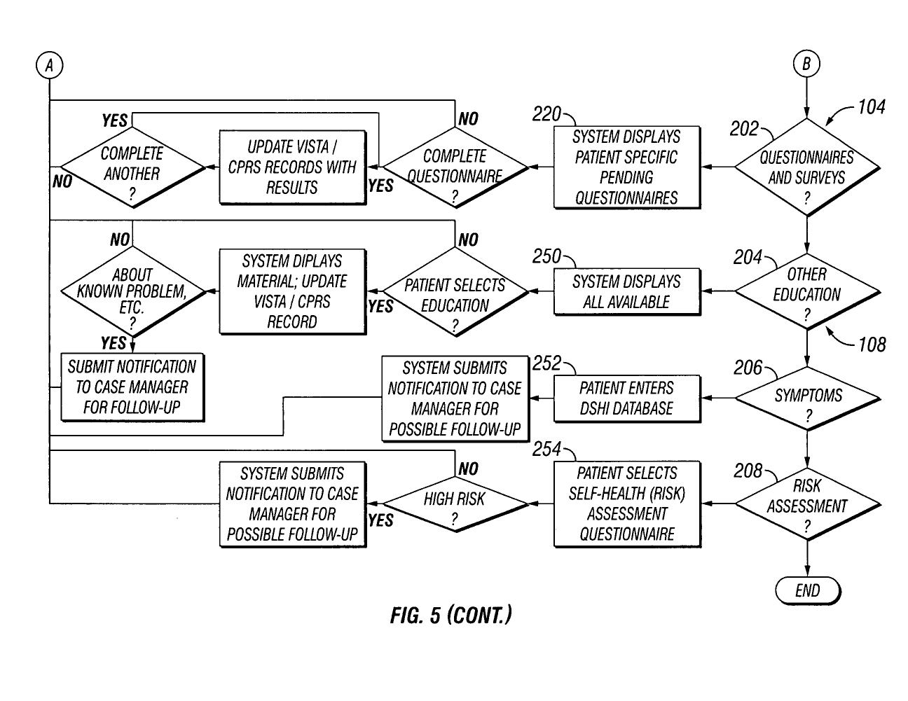

Specifically, referring to FIGS. 2 and 5, at Loop-D, once a patient successfully logs into the patient specific application domain, system 100 may retrieve information specific to the patient according to the signed release of information authorization at block 192. At block 194, the patient may be presented buttons that represent the categories of information that the system is authorized to display by the release of information. Touching a button serves as a portal to specific applications at block 196. For example, touching the appropriate button at block 196 provides a patient access to medical record information application at block 198, appointment information application at block 200, questionnaire and survey information application at block 202, education information application at block 204, symptom information application at block 206 and risk assessment information application at block 208.

With regard to medical record information application at block 198, a patient may be provided with access to limited extracts from their medical records. For example, in a survey conducted in 2001, patients were asked questions such as, did you get answers you could understand, did provider explain need for tests in an understandable way, did provider explain test results in understandable way, did someone explain prescriptions in understandable way, did you know the next step in your care, etc. In responding to these questions, more than thirty percent of the average respondents answered the questions negatively. In order to overcome the aforementioned drawbacks with patient access to limited extracts from their medical records, medical record information block 210 may include a problem list with links to education material, medication list with links to education material, recent laboratory result with links to education material and pending orders with links to preparation material. Specifically, the problem list with links to education material may display and link a patient's problem list entries to educational material specific to the entry. The medication list with links to education material may display and link the patient's active medication list to pharmacy approved educational material. The recent laboratory result with links to education material may display recent laboratory results and link them to appropriate educational material. The pending orders with links to preparation material may display pending orders, scheduled visits and patient preparation instructions. A patient may also have the ability to obtain refills at block 212 with an automatic order being placed to a pharmacy at block 214.

With regard to appointment information application at block 216, amongst other benefits discussed below, a patient may be able to look up future appointments without having to call their specific healthcare provider. For example, communicating initial appointments and changes to patients is a massive undertaking. In a single year in which patients can make hundreds of thousands of appointments at a single healthcare provider, initial appointments and changes entail a unique communication effort as scheduling efforts at the healthcare provider are not currently synchronized. Mailed appointments can routinely take two or more weeks to reach patients. Consequently, patients regularly show up for their appointments on the wrong day, and thousands of mailed appointments are returned annually due to incomplete or missing addresses. The patients who don't show up waste inordinate amounts of healthcare provider resources and contribute to prolonged waits and delays. Most providers have no show rates that approach 20%, while some have rates as high as 30%. Strategies for reducing no shows such as calling patients two days before an appointment, while effective, require significant staffing resources.

In order to obviate some of the aforementioned problems with appointment setting methodologies used by most healthcare providers, appointment information application at block 216 may allow patients to look up their future appointments, as well as permitting patients to cancel or request appointment rescheduling. Appointment information application at block 216 may also enable patients to review and request changes to their appointments at block 218. Appointment information application at block 216 would most likely impact frequent patients, by providing them direct access to their future appointment schedules, thereby reducing their dependency on an unreliable appointment mailing system and telephone reminders and inquiries.

With regard to questionnaire and survey information application at block 220, a patient may be prompted to answer and enter data directly into his or her records, without an intermediary healthcare professional entering such data. Since patients are often asked by their healthcare provider to update their medical record, often more than once a year, questionnaire and survey information application at block 220 will allow patients to update their medical record appropriately and clear any outstanding reminders requiring patients to update records. Additionally, compared to paper based questionnaires, questionnaire and survey information application at block 220 will also allow patients to skip unneeded follow-up questions, which have been answered during previous sessions.

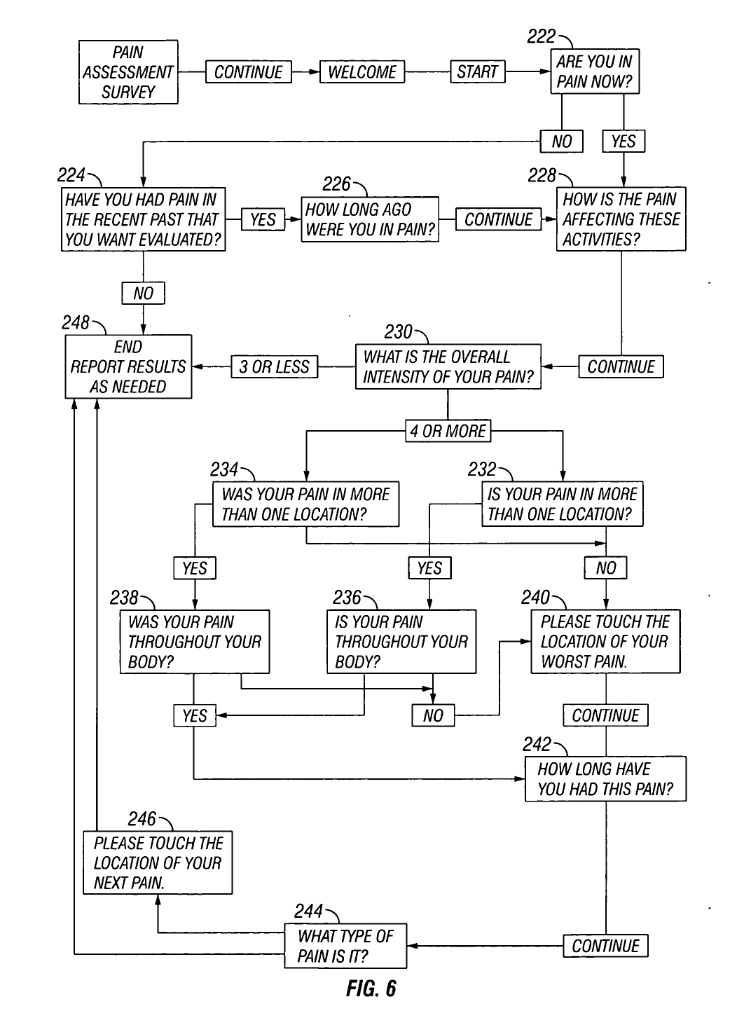

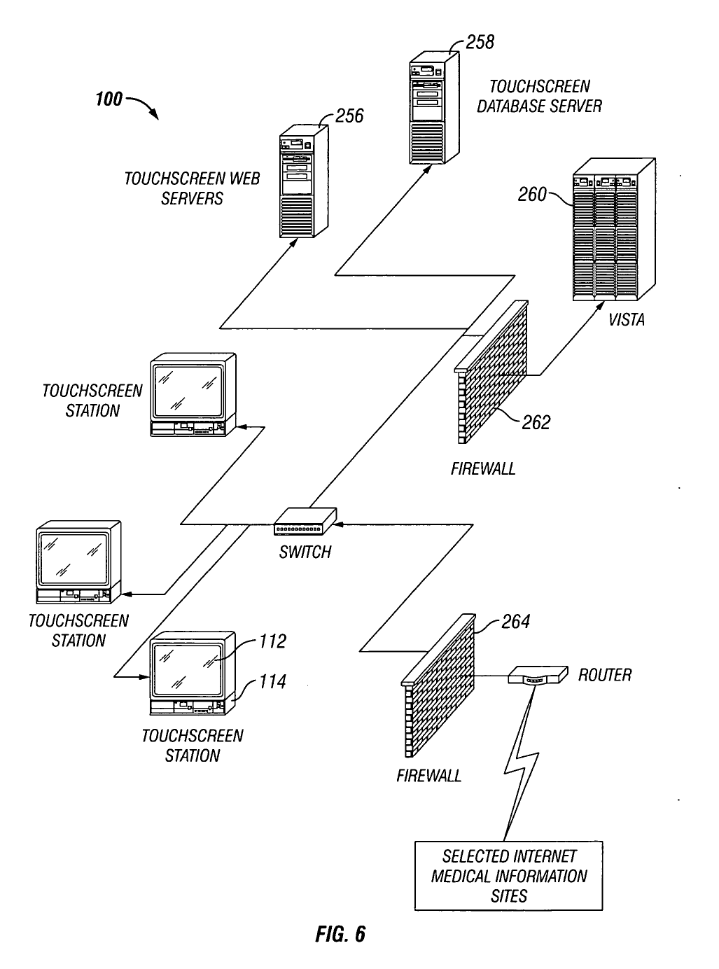

As discussed above and as illustrated in FIGS. 5 and 6, questionnaire and survey information application at block 220 may also provide for pain assessment. For a patient experiencing pain, questionnaire and survey information application at block 220 may prompt a patient with a sequence of questions such as, are you in pain now (block 222), have you had pain in the recent past that you want evaluated (block 224), how long ago were you in pain (block 226), how is the pain affecting these activities (block 228), what is the overall intensity of your pain (block 230), is your pain in more than one location (block 232), was your pain in more than one location (block 234), is your pain throughout your body (block 236), was your pain throughout your body (block 238), please touch the location of your worst pain (block 240), how long have you had this pain (block 242), what type of pain is it (block 244), please touch the location of your next pain (block 246), and report the results (block 248). For block 240, a patient may be prompted to locate the pain they are experiencing on a figurine (not shown) provided on screen 112. A patient that touches a specific area on the figurine may then be asked additional questions, such as those identified in blocks 242 and 244 that may help identify the cause. For example, a patient that points to a knee may be asked questions that relate to activity and rest, while a patient that touches the head may be asked questions that characterize headaches.

As discussed above, questionnaire and survey information application at block 220 may also provide for means testing for patients who are required to have a means test on file to receive care at healthcare providers. Such tests may be automatically administered and transmitted using questionnaire and survey information application at block 220. Accordingly, registered patients may access the touch-screen application system 100, answer the necessary questions, and then “sign” the completed form using a signature pad (not shown). Lastly, questionnaire and survey information application at block 220 may yet further be used for measuring healthcare effectiveness, using (i.e. by administering SF-36 forms for veterans).

Referring to FIG. 5, upon accessing education information application at block 250, a patient may obtain access, for example, to non-problem list medical conditions with links to education material. Specifically, non-problem list medical conditions with links to education material may provide links to a wide variety of medical conditions and general medical information, and point to healthcare provider developed educational materials as well as selected reliable Internet sites.

With regard to symptom information application at block 252, patients logging into the secure, patient-specific domain of symptom information application at block 252 may have access to software that provides symptom triaging. Outcomes may point to relevant educational material.

With regard to risk assessment information application at block 254, patients logging into the secure patient-specific domain of risk assessment information application at block 254 may have access to a number of health-screening tools, with outcomes pointing to relevant educational material.

Referring to FIG. 7, for security and for ease of maintenance, touch-screen application system 100 may be developed on dedicated servers 256, 258, and workstations 114 having touch-screens 112 using web based applications that operate separately from an healthcare provider's clinical information and financial systems. In the exemplary embodiment of FIG. 7, the architecture may consist of at least two servers, a web application server 256 and a patient database server 258, as well as sufficient workstations 114 to accommodate workload. Specific application software may reside on web application server 256. All applications may be developed using web-based protocols and information software. Redacted patient information may reside on the touch-screen database for registered patients. This information may be updated and deleted in accordance to release of information authorization. Touch-screen database server 258 may communicate with VISTA system 260 though a firewall 262. Messaging protocols, such as HL-7, may be used to transfer selected patient record information from VISTA system 260 to touch-screen database server 258 and to transmit questionnaire and education documentation from touch-screen sessions to VISTA system 260. Patient access to selected Internet sites may be controlled through web application server 256. Applications may limit patient access to healthcare provider approved sites and may limit their ability to follow URL links to other, non-approved, sites. All Internet access may pass through a dedicated firewall 264.

Touch-screen units (workstations) 114 may be designed for ease of use by patients while ensuring privacy and security. Each unit 114 may consist of a PC, a touch-screen monitor with touch screen 112, a printer (not shown), a patient identification card reader (not shown), a fingerprint reader (not shown), and a signature pad (not shown). Since all operations may be controlled by touch screen 112, there may be no keyboard or mouse attached to the unit. All hardware components may be built into a locked, desk-like enclosure with only the user interface components accessible. The enclosures may be designed to be wheelchair accessible and placed in such a manner that screens are not visible by casual observers.

The aforementioned applications for touch-screen application system 100 may be developed in a modular fashion.

Although particular embodiments of the invention have been described in detail herein with reference to the accompanying drawings, it is to be understood that the invention is not limited to those particular embodiments, and that various changes and modifications may be effected therein by one skilled in the art without departing from the scope or spirit of the invention as defined in the appended claims.

What is claimed is:

1. A method for outpatient process automation, said method comprising:
prompting a user to select at least one anonymous or patient specific application;
if the user selects said anonymous application, receiving from the user anonymous query information, matching said anonymous query information to anonymous information stored in a database, and displaying guidance based upon said matched anonymous information; and
if the user selects said patient specific application, receiving from the user patient specific query information, matching said patient specific query information to patient specific information stored in a database, and displaying guidance based upon said matched patient specific information,
wherein user access to said patient specific application being controlled by a secure log-in process.

2. A method according to claim 1, wherein said anonymous application including at least one of way-finding information, general healthcare provider information, eligibility and benefits information, and staff directory information for healthcare providers.

3. A method according to claim 1, wherein said secure log-in process for access to said patient specific application comprising:
prompting the user to log-on by entering a social security number by either touching a displayed number pad or sliding an identification card through an attached card reader, and prompting the user to enter a password;
if said log-in being successful, providing the user access to said patient specific application; and
if said log-in being unsuccessful, assisting the user to obtain access to said patient specific application.

4. A method according to claim 3, wherein said assisting including:
verifying that a system for implementing said method being operational;
if said system being operational and the user is a registered user, determining whether the user password and identification card are operational, if not, then assisting the user to obtain a new password or identification card, if so, then releasing requested information to the user;
if said system being operational and the user is a non-registered user, assisting the user to log-in and register into said system by means of the social security number; and
if said system being non-operational, moving the user to another system for implementing said method.

5. A method according to claim 3, wherein said providing including:
controlling release of patient information in accordance with HIPAA regulations by requiring the user to authorize display of patient specific application data through a signed release of information (ROI).

6. A method according to claim 5, wherein said providing including;
prompting the user to select at least one of a medical record information application, an appointment information application, a questionnaire and survey information application, an education information application, a symptom information application, and a risk assessment information application.

7. A method according to claim 6, wherein said medical record information application including at least one of a problem list with links to education material, medication list with links to education material, recent laboratory results with links to education material, pending orders with links to preparation material, and means for enabling a user to order medication refills.

8. A method according to claim 6, wherein said appointment information application enabling the user to at least one of look up and change future appointments for a healthcare provider.

9. A method according to claim 6, wherein said questionnaire and survey information application enabling the user to at least one of answer and enter data directly into said system, enabling means testing and enabling measurement of healthcare effectiveness.

10. A method according to claim 6, wherein said education information application enabling the user to obtain information on at least one of medical conditions and general medical information.

11. A method according to claim 6, wherein said symptom information application enabling the user access to software that provides symptom triaging.

12. A method according to claim 6, wherein said risk assessment information application enabling the user access to health-screening tools.

13. A system for outpatient process automation, said system comprising:
means for prompting a user to select at least one anonymous or patient specific application;
means for selecting said anonymous application;
means for selecting said patient specific application; and
means for controlling access to said patient specific application by a secure log-in process,
wherein, for said anonymous application, said system receiving from the user anonymous query information, matching said anonymous query information to anonymous information stored in a database, and displaying guidance based upon said matched anonymous information, and for said patient specific application, said system receiving from the user patient specific query information, matching said patient specific query information to patient specific information stored in a database, and displaying guidance based upon said matched patient specific information.

14. A system according to claim 13, wherein said anonymous application including at least one of way-finding information, general healthcare provider information, eligibility and benefits information, and staff directory information for healthcare providers.

15. A system according to claim 13, wherein said secure log-in process for access to said patient specific application comprising:
means for prompting the user to log-on by entering a social security number by either touching a displayed number pad or sliding an identification card through an attached card reader, and means for prompting the user to enter a password;
if said log-in being successful, means for providing the user access to said patient specific application; and
if said log-in being unsuccessful, means for assisting the user to obtain access to said patient specific application.

16. A system according to claim 15, wherein said means for assisting including:
means for verifying that said system being operational;
if said system being operational and the user is a registered user, means for determining whether the user password and identification card are operational, if not, then assisting the user to obtain a new password or identification card, if so, then releasing requested information to the user;
if said system being operational and the user is a non-registered user, means for assisting the user to log-in and register into said system by means of the social security number; and
if said system being non-operational, means for moving the user to another system for implementing said method.

17. A system according to claim 15, wherein said means for providing including:
means for controlling release of patient information in accordance with HIPAA regulations by requiring the user to authorize display of patient specific application data through a signed release of information (ROI).

18. A system according to claim 17, wherein said means for providing including;
means for prompting the user to select at least one of a medical record information application, an appointment information application, a questionnaire and survey information application, an education information application, a symptom information application, and a risk assessment information application.

19. A system according to claim 18, wherein said medical record information application including at least one of a problem list with links to education material, medication list with links to education material, recent laboratory results with links to education material, pending orders with links to preparation material, and means for enabling a user to order medication refills.

20. A system according to claim 18, wherein said appointment information application enabling the user to at least one of look up and change future appointments for a healthcare provider.
